(19) United States
(12) Patent Application Publication
(10) Pub. No.: US 2006/0122572 A1
(43) Pub. Date: Jun. 8, 2006

(54) Transfer layer of liquid fluids and an absorbent article incorporating the same

(75) Inventor: Claudio Miguel Suarez, PROVINCIA DE BUENOS AIRES (AR)

Correspondence Address: DAVIDSON, DAVIDSON & KAPPEL, LLC, 485 SEVENTH AVENUE, 14TH FLOOR, NEW YORK, NY, 10018, US

(73) Assignee: CARL FREUDENBERG KG, WEINHEIM (DE)

(21) Appl. No.: 10/517,331

(22) PCT Filed: Jun. 4, 2003
(86) PCT No.: PCT/EP03/05848

§ 371 Date: Dec. 9, 2004

(30) Foreign Application Priority Data
Nov. 6, 2002 (AR) P02 01 002203

(51) Int. Cl. A61F 13/15

(52) U.S. Cl. 604/385.101; 604/378

(57) ABSTRACT

A transfer layer of liquid fluids and an absorbent article incorporating the transfer layer, like a diaper, a sanitary napkin or a similar product, which has a permeable cover below which there is disposed a nonwoven transfer layer for liquid fluids followed beneath by an absorbent core which retains the fluids. In the transfer layer there is provided a top layer of predominating hydrophobic properties and a bottom layer of predominating hydrophilic properties. The transfer layer has an embossed surface configuration, with channels formed by compressed nonwoven streaks forming transversal peaks and valleys extending in longitudinal direction along the transfer layer. The lower thickness of the transfer layer resulting from the compressed stretches which form valleys, provide the channels with a predominating hydrophilicity, which facilitates the quick liquid transfer towards the absorbent core, while the higher thickness of the transfer layer in the peaks provide it with a predominating hydrophobia, which impedes the liquid to return from the absorbent core to the permeable cover, thus reducing the index of remnant humidity in the zone of contact with the user's skin, even if the absorbent core is submitted to pressure.

The present invention refers to a transfer layer of liquid fluids and absorbent articles with transfer layer inside, like diapers, sanitary napkins or similar products. More precisely, the article that is part of the present invention is of that type which includes a layer permeable to fluids and destined to be in contact with the user's skin, beneath which there is a transfer layer of liquid fluids followed by an absorbent core which receives these liquids and retains them in order to avoid the contact with the user's skin while using the article. The transfer layer used in this type of articles is generally made of a nonwoven conformed by fibers which use to have very similar properties, although they differ slightly among one another.

It is known that the function of the permeable cover is to permit the passage of the liquid. Some of the prerequisites of this coversheet is its smooth skin contact and mechanical resistance and, moreover, it is required to remain as dry as possible even after the passage of a great quantity of liquid. The absorbent core for its part requires not only a high capacity and speed of absorption but also an efficient liquid retention, even after having been submitted to pressure during the normal use of the article in question.

Regarding the transfer layer it must fulfill specific special functions. In effect, whatever be the structure of this transfer layer, in any of the different alternatives of application, it is determined to improve the pass-thru speed of the liquids, diminish the drain risk, reduce the superficial humidity to which the user's skin is exposed after the absorption of the liquid, improve the liquid distribution in order to benefit to the greatest possible extent from the core's absorption and retention capacity.

### FOREGOINGS OF THE INVENTION

Experts in this matter know that most of the transfer layers used in diapers, sanitary napkins etc. are made of homogeneous nonwovens whose structure is constituted of the same fiber type at the same proportion. However, transfer layers may be found which present progressive porimetrical sizes from one side to the other according to their production process. Both surfaces of the top and bottom side of these transfer layers are plain; therefore the surface of the top layer is always in complete contact with the surface of the permeable layer in front of it. Consequently, the fluid transfer through the nonwoven's structure of the transfer layer is always homogeneous and capillary. The state of art for absorbent products require necessarily a transfer layer with hydrophilic properties to allow the liquid to pass through, which from its concept is a contradiction, as a hydrophilic surface in full contact with the permeable layer cannot efficiently isolate the humidity from the absorbent core. It must be pointed out that it has been proved that the remnant humidity of the liquid is transmitted in a capillary way from the absorbent core to the permeable layer.

One of the solutions found in some habitual articles consists in increasing the thickness of the transfer layer. However, practice has shown that this solution does not lead to acceptable results unless the weight per area unit of the transfer layer is increased, which unfortunately results in a considerable cost increment of the transfer layer for most applications.

There have been other solutions to solve the problems mentioned before, which can be found in the North American Patent Nr. U.S. Pat. No. 6,245,901 from SCA HYGIENE PRODUCTS AB which refers to an absorbent article like baby diapers, adult incontinence diapers, sanitary napkins, bandages and similar products. This article is constructed with a layer of continuous fibers, frequently called “TOW”, bonded among each other at distinct points, lines or sectors according to a pattern, while in other parts they are not bonded. Said layer comprises at least two zones as becomes visible in a transversal cut of the layer. These zones are different regarding one or various properties as for instance basic weight, density, pore size, hydrophillic/hydrophobic property and/or other absorption properties and/or properties which affect the skin conditions. This layer can also be utilized as a liquid receiving layer which is hidden below the cover like a cover or also like a combination of cover and liquid receiving layer. Although the absorbent article published in this US patent presents certain structural differences concerning the design of the transfer layer, it is not expressly stated that the afore-mentioned problems are solved by referring to the structure of the conventional absorbent articles and particularly referring to the passage of the liquid fluids through the transfer layer. The figures annexed to the description of this US patent clearly show that the transfer layer has a great number of sectors where the top side is adhered to the bottom side in different configurations which in any case define specific zones of liquid transfer, depending on the configuration which adopt said unions between the mentioned top and bottom side.

FIG. 4 of such patent, shows that the bonding method provided in the transfer layer have a pattern of many straight paths with an alternating orientation among each other and a transversal orientation with respect to the longitudinal orientation of the fibers which the transfer layer is made of. Although the unions between the top and bottom layer of the transfer layer may adopt different configurations, i.e. a pattern of alternating straight segments mainly of the same length, as shown in FIG. 4 of this US patent, or many straight segments conformed in a criss-cross pattern or many points or sectors of union which are not expressly illustrated in this US Patent.

From the description and illustration of this absorbent article it must be concluded that the segments and points or sectors of union constructed in the transfer layer serve the purpose of consolidating or reinforcing the internal structure formed by the fibers which the transfer layer is composed of. It is worth mentioning that whatever configuration be adopted it does not contribute to improving the capacity of absorption and transfer of the liquids which pass through the absorbent layer and must be retained in the absorbent core. Consequently, apart from an improvement of certain structural aspects of the transfer layer, the absorbent article as proposed by the U.S. Pat. No. 6,245,961 does not succeed in solving the inconveniences commented before when generally referring to the conventional absorbent articles. Moreover, for the diverse alternatives described and illustrated, this US Patent proposes variants where, however, the transfer layer's structure maintains the same preferentially commented specific peculiarities. Therefore, they do not solve the deficiencies of this type of transfer layer either, which is clearly shown by the figure which illustrates a usual absorbent product that is part of the present description.

### SUMMARY OF THE INVENTION

The transfer layer and the absorbent article which is part of it according to the present invention, was originate from the need to improve the cost-benefit relation of the articles based on standard technology. It can achieve a higher speed of the liquid transfer and/or diminish considerably the residual humidity in the transfer layer, thus assuring the user in any circumstance of use of this absorbent article to keep the skin dry. To this end, the absorbent article according to the present invention offers advantageous properties that result from three innovative concepts which focus on a combination among each other in order to obtain the proposed objective.

The first concept consists in providing a transfer layer with embossed channels on top layer, i.e. the face of the transfer layer which is in contact with the topsheet is provided with a number of channels that are oriented in the same direction and whose depth and separation may be changed in the production of the article according to the type of use it is destined for. To give one practical example, a transfer layer may be constructed with channels of 6 mm width at a distance of also 6 mm. In this way, the embossed surface provides the contact with the permeable layer and determines two clearly different zones below the permeable cover, i.e. a succession of fibrous zones which are separated by the corresponding channels that produce longitudinal air spaces. In trials it has been proved that these air channels have a very favorable influence on the fluid's transfer speed as they are not conducted through the channels in a capillary way.

The second concept refers to the structure of different layers. As a matter of fact, in contrast to the known absorbent articles, the transfer layer in the product of the present invention has a multilayer structure with two very different layers: one hydrophobic top layer which maintains partial contact with the absorbent cover of the article and one hydrophilic bottom layer which has plain contact with the absorbent core, whereby this bottom layer has preferably a permanent hydrophilicity.

From the figure shown later to illustrate the present invention it becomes clear that thanks to the special composition of the transfer layer, the top (hydrophobic) layer of the nonwoven does not allow the fluids flow opposite to the desired direction, i.e. from the absorbent core to the top layer or, finally to the permeable cover. This functional property together with the reduced contact surface between the top layer and the permeable cover makes it possible to reach considerably lower superficial humidity values even if the nonwoven has a low weight per area unit. This characteristic can be called “valve effect”, as it conditions the fluid transfer to take only one single direction, i.e. from the permeable cover towards the absorbent core.

The third concept refers to the relation between the variable density and the variable hydrophobicity in the transfer layer, which can be considered the most important concept as it permits the fluid to be quickly absorbed by the absorbent core.

Nevertheless, it is worth mentioning that if the hydrophobic top layer was highly hydrophobic, the fluid would run through the channels displacing itself on their surface along the extension defined by the transfer layer's structure, but without being able to be transferred towards the absorbent core, which without any doubt is not the desired or expected result. This situation is taken into consideration and efficiently resolved thanks to the special characteristics of the transfer layer and the absorbent article according to the present invention.

If the hydrophobicity of the hydrophobic top layer is relatively low, e.g. with a Basket Test (ASTM D-1117-5.2) of 25 seconds, the zones where the fibers have not been compressed, namely at both sides of every longitudinal channel, remain sufficiently dry during the period of the article's use. However, in the channels with a density of over 0.3 g/cm3, the hydrophilicity of the bottom layer of the transfer layer is the predominant variable, permitting an efficient conduction of the liquids through the nonwoven. As is shown later referring to the corresponding figures, by making a transverse cut in the transfer layer, a nonwoven structure becomes visible in which hydrophobic parts are found in the zones of highest thickness or “mountains” and hydrophilic parts in the zones of lowest thickness or “valleys” of said transfer layer.

In lab trials by means of comparative techniques between the present invention and the habitual products, it was possible to observe transfer speeds which were approximately up to 30% higher with the same weight per area unit, while the remnant humidity in the permeable cover was considerably lower, even with up to 40% lesser weights per area unit.

Thus it is the aim of the present invention to provide an article for the absorption and retention of liquid fluids, as a diaper, a sanitary napkin or similar product, which is constructed with a permeable cover below which there is a nonwoven transfer layer and beneath that an absorbent core that retains the fluids. In the transfer layer there is a chiefly hydrophobic top layer and a chiefly hydrophilic bottom layer. The transfer layer presents a embossed surface with channels conformed by compressed parts of the nonwoven, hereby developing peaks and valleys in transverse pattern which extend in machine direction along the transfer layer. Thanks to the minor thickness of the transfer layer in the compressed parts which are the valleys, the channels adopt hydrophilic properties which facilitates the quick liquid transfer to the absorbent core. Thanks to the major thickness of the transfer layer in the peaks, hydrophobic properties are achieved which avoid the liquid return from the absorbent core to the permeable cover. It reduces the index of remnant humidity in the zone of skin contact even if the absorbent core is undergoing pressure.

Therefore it is the objective of the present invention to provide an article for the absorption and retention of liquid fluids, as e.g. a diaper, a sanitary napkin or similar product. This type of article includes a permeable cover which is in contact with the user's skin, below which there is a layer for the liquid transfer followed by an absorbent core to retain the fluids, whereby the mentioned transfer layer is made of nonwovens, its structure comprising one top layer of fibrous material characterized by predominating hydrophobicity and at least one bottom layer of predominating hydrophilic properties, united with each other in parts that form channels of distribution and transfer of liquids towards the absorbent core, forming peaks between every pair of adjacent channels where both superposed layers have a higher transversal thickness, while every channel formed between adjacent peaks define a valley where both layers have a lower transversal thickness.

### BRIEF DESCRIPTION OF THE FIGURES

For a better comprehension of the object of the present invention it is illustrated by various figures which show the article in the preferential type of realization, only for the sake of giving an example.

FIG. 1: It is a sketch which gives a partial view of a transverse cut. It shows the structural conformation of an absorbent article according to one of the examples of the previous articles.

FIG. 2: It is a sketch which gives a partial view of a transverse cut. It shows the structural conformation of the absorbent article of the invention in question.

FIG. 3: It is a sketch which gives a partial view of a transverse cut. It shows the structural conformation pertaining to the absorbent article according to the invention in question, indicating by arrows the flow of the liquids which enter by the permeable cover and are absorbed by the absorbent core through the transfer layer.

FIG. 4: It is a sketch which gives a perspective and cut view of the transfer layer's structure pertaining to the absorbent article according to the present invention.

### DETAILED DESCRIPTION OF THE INVENTION

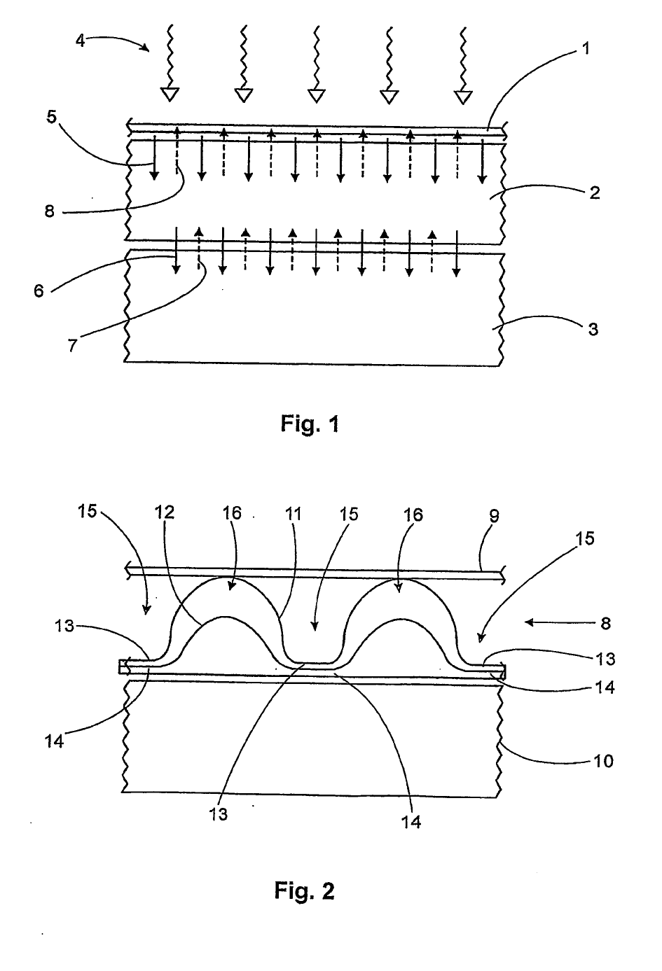

FIG. 1 depicts the absorbent article according to the previous art. It is comprised of a permeable cover 1 beneath which there is a transfer layer 2 followed immediately by an absorbent core 3. These elements have been plotted with small separations between one another to ensure a clearer visualization of sketch 1. The meandered arrows 4 represent the liquid fluids which flow on the exterior surface of the permeable cover 1. The straight arrows 5 show the passage of the fluid through the transfer layer 2 while arrows 6 mark the fluid leaving the transfer layer 2 and being absorbed by the absorbent core 3. It may be observed that the complete surfaces of cover 1 and transfer layer 2 are in plain contact with one another, so that the liquid 4 can pass through any of the points of contact, and the same occurs to the surfaces of transfer layer 2 and the absorbent core 3. In the previous design of the article—as commented before—in fact the fluid transfer is homogeneous and capillary throughout the whole nonwoven's structure of the transfer layer 2. The hydrophilic property of the transfer layer and its plain contact with both the cover 1 and the absorbent core 3, allow the fluid to return in a capillary manner from core 3 to cover 1 through the transfer layer 2 as marked by the streaked arrows 7. It has been said before that this inconvenience may be solved by increasing substantially the thickness of the transfer layer 2. As a matter of fact, it would be possible to obtain acceptable results by incrementing the weight per area unit, but this would boost the cost of the transfer layer 2 in many applications of the article and additionally imply other practical inconveniences and disadvantages for the user of these products.

FIG. 2 shows the present invention. It comprises a transfer layer 8 placed between a permeable cover 9 and an absorbent core 10. The cover 9 and absorbent core 10 may be the same as those used in habitual absorbent articles. The transfer layer 8 is composed of a top layer 11 made of a predominating hydrophobic fibrous material and an bottom layer 12 made of a predominating hydrophilic fibrous material.

The layers 11 and 12 are united with one another in the parts 13 and 14 which altogether form channels 15, also called “air channels”, for the distribution and transfer of the liquid that passes through the permeable cover 9 and which finally is absorbed by the core 10.

Between every pair of adjacent channels 15, a “peak” 16 is formed, where the two layers 11 and 12 have a major transversal thickness. The smallest thickness of both layers is is area 13 and 14, where every channel 15 forms a valley between each pair of adjacent peaks 16.

The thickness of the top layer 11 of the transfer layer 8 diminishes progressively from the peaks 16 to the valleys that define every channel 15. The thickness values which will be mentioned in the following refer to the visual thickness which can be observed in relaxed conditions, e.g. in a profile projector or similar device. The thickness of the transfer layer 8 in the valleys is defined by the weight per area unit and the density to which the two layers are compressed. This thickness is provided to be between 0.10 mm, corresponding to a weight of 30 g/m2 and a density of 0.30 g/cm3, and 0.40 mm, corresponding to a weight of 80 g/m2 and a density of 0.20 g/cm3. The thickness of the transfer layer 8 in the peaks 16 is defined by the depth of the cylinder or device used to emboss the channels on the nonwoven, the same having a maximum height of 10 mm.

According to the variation of the thickness, the density of the nonwoven's fibers at the peaks 16 is lower than the fiber density at the parts 13 and 14, where there are the channels 15 in which the transfer layer has its major liquid transfer capacity. For the valve effect at least two different densities are provided, although maximum and minimum density values can be reached, not only by continuous density variation but also by an abrupt jump, in both cases achieving the valve effect. The maximum density in the valleys can reach a value of up to 0.35 g/cm3, and may be lower but not less than 0.18 g/m3, to an extent which according to the hydrophobic properties of the top layer allows the liquid passage. The density of the transfer layer 8 in the valleys has practically an even higher range and is defined by the embossing of the channels and fibers used with characteristic values between 0.03 g/cm3 and 0.2 g/cm3. Owing to these structural properties of the transfer layer 8, the top layer 11 registers a lower coefficient of hydrophobicity in the channels 13 and 14 than in the peaks 16. A practical way to corroborate this phenomenon is to put softly one drop of an aqueous 0.9% solution of chloride of sodium (NaCl). The test shows that the drop humidifies the surface in the valleys, settling in the structure of the transfer layer 8, while the drop does not humidify the transfer layer 8 in the peaks and stays on the surface.

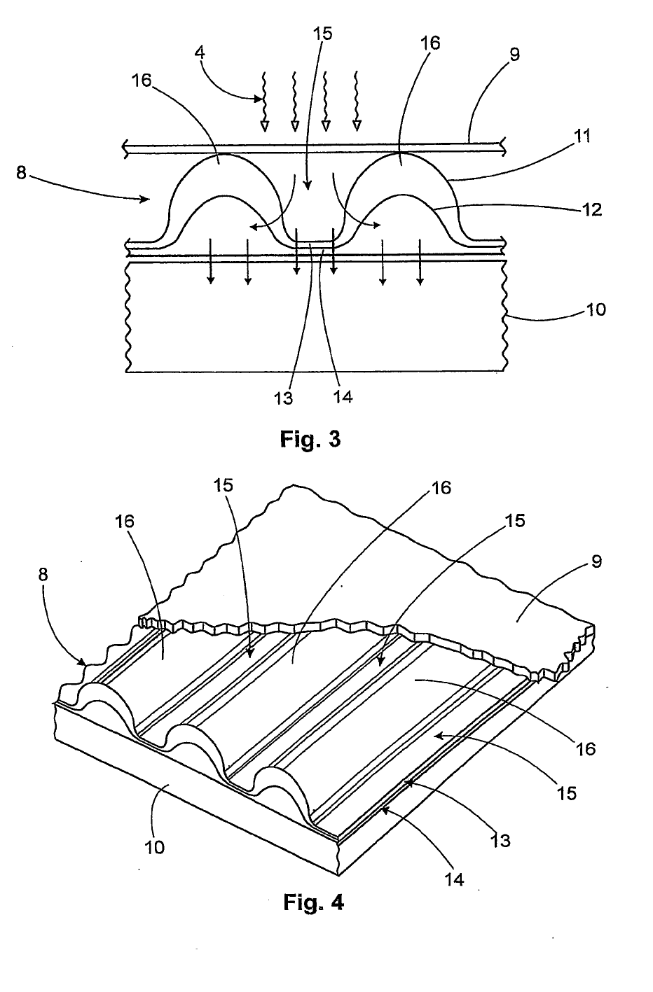

As is shown in FIG. 3, in this way the peaks 16 define zones of superficial distribution of liquids towards the mentioned valleys defined by the channels 15. As a matter of fact, FIG. 3 depicts clearly the liquid flow represented by the arrows 4 after passing through the permeable cover 9. As the thickness of the layer 11 in the peaks 16 has a higher hydrophobia than in the compressed portion which forms the channel 13, the liquid can only pass through the transfer layer 8 when it encounters a lower thickness in the layer 11, which is in the zones adjacent to the channel 13 and 14 where the hydrophilicity is predominant. Consequently, the major portion of the liquid transfer to the absorbent core 10 is quickly reached through the channel 13 and 14 of the channels 15 and a lesser portion through the zones of the transfer layer immediately adjacent to the mentioned channel 13 and 14.

Once the liquid is absorbed, distributed and retained in the bosom of the absorbent core 10, and even if it should happen that part of the liquid flows towards the hydrophilic layer 12, the hydrophobic property of the layer 11 is sufficient to impede the liquid to return to the surface of this layer 11. As the transfer layer 8 offers less contact surface to the cover, only 40% to 80% of the surface in conditions of use—and this surface posses a certain grade of hydrophobicity—render difficult the capillary transmission of humidity from the absorbent core to the cover, even under pressure.

Thus it can be confirmed that the union of the top layer 11 and the bottom layer 12 results in a transfer layer 8 which allows the transfer of liquids in only one direction, i.e. from the permeable cover 9 to the absorbent core 10.

Regarding the design for printing the channels, e.g. a sinusoid pattern may be considered which permits to change progressively the density additionally rendering a pleasant touch. Nevertheless, the present invention contemplates also other profiles which possess two or more levels and permit to change the density, as for instance cornered (square), toothed or trapezoid curves.

According to this conception, the dimensions of the channels 15 and the peaks 16 are extremely variable also considering the esthetic aspect of the transfer layer. The present invention comprises any shape of channel with peaks and channels of more than 1 mm width.

FIG. 4 which gives a perspective view clearly illustrates the general aspect of the transfer layer's structure 8 which is composed of the layers 11 and 12 that are disposed between the permeable cover 9 and the absorbent core 10, and particularly the conformation of the channels 15 and peaks 16.

1-10. (canceled)

11. A transfer layer for a liquid fluid and for application in an absorbent article of personal use, the transfer layer comprising:
a top layer of predominantly hydrophobic fibrous material;
a bottom layer of predominantly hydrophilic material superimposed on the top layer and joined to the top layer at a plurality of joining regions of the top and bottom layers so as to form a plurality of channels at the joining regions, a plurality of peaks being formed of the top and bottom layers between adjacent ones of the plurality of channels, wherein a transversal thickness of the top and bottom layers is lower at the joining regions than at the peaks.

12. The transfer layer as recited in claim 11, wherein the plurality of peaks define zones of superficial liquid distribution to the channels.

13. The transfer layer as recited in claim 11, wherein a hydrophobia of the top layer and a hydrophilicity of the bottom layer are such that the liquid fluid is transferred through the top layer to the bottom layer at the channels.

14. The transfer layer as recited in claim 13, wherein the hydrophobia of the top layer and the hydrophilicity of the bottom layer are such that the liquid fluid is impeded from transferring through the top layer to the bottom layer at the peaks.

15. The transfer layer as recited in claim 14, wherein a thickness of the top layer is between 0.6 and 10 mm at the peaks and between 0.1 and 0.4 mm at the joining region of the top layer.

16. The transfer layer as recited in claim 11, wherein a fiber density of the transfer layer in the peaks is lower than the fiber density at the joining regions.

17. The transfer layer as recited in claim 16, wherein the fiber density of the transfer layer in the peaks is between 0.03 and 0.2 g/cm3 and the fiber density at the joining regions is between 0.18 and 0.35 g/cm3.

18. The transfer layer as recited in claim 11, wherein the transfer layer has a lower hydrophobia at the joining regions than at the peaks.

19. The transfer layer as recited in claim 11, wherein a coefficient of hydrophobia in the top layer decreases from the peaks to the channels.

20. The transfer layer as recited in claim 11, wherein the top layer and the bottom layer provide for a unidirectional flow of the liquid fluid.

21. The transfer layer as recited in claim 11, wherein the article includes one of a diaper, a sanitary napkin and a bandage.

23. The transfer layer as recited in claim 11, wherein the plurality of channels are arranged in parallel lines.

23. The transfer layer as recited in claim 11, wherein each of the plurality of channels form a linear region of liquid distribution and transfer.

24. An article for the absorption and retention of a liquid fluid, comprising:
a cover permeable to fluids and configured to be in contact with a user's skin;
a transfer layer provided below the cover, the transfer layer including a top layer of predominantly hydrophobic fibrous material and a bottom layer of predominantly hydrophilic material superimposed on the top layer and joined to the top layer at a plurality of longitudinal joining regions of the top and bottom layers so as to form a plurality of channels at the joining regions, a plurality of peaks being formed of the top and bottom layers between adjacent ones of the plurality of channels, wherein a transversal thickness of the top and bottom layers is lower at the joining regions than at the peaks, wherein the channels are in contact with the cover;
an absorbent core configured to absorb and retain the liquid fluid.

25. The article as recited in claim 24, wherein the transfer provides a unidirectional liquid transfer layer for passing the liquid fluid through the permeable cover to the absorbent core.

26. The article as recited in claim 24, wherein the article includes at least one of a diaper, a sanitary napkin, and a bandage.

27. The article as recited in claim 24, wherein each of the plurality of channels forms a linear region of liquid distribution and transfer.
